(19) United States
(12) Patent Application Publication
(10) Pub. No.: US 2023/0320621 A1
(43) Pub. Date: Oct. 12, 2023

(54) WEARABLE BIOMETRIC SENSOR

(71) Applicant: X Development LLC, Mountain View, CA (US)

(72) Inventor: Samuel Khamis, San Francisco, CA (US)

(21) Appl. No.: 17/716,844

(22) Filed: Apr. 8, 2022

(51) Int. Cl. A61B 5/083; A61B 5/00

(52) U.S. Cl. CPC A61B 5/0836; A61B 5/681; A61B 5/0833; G06N 3/02; A61B 2562/0247; A61B 2562/028; A61B 2560/0475

(57) ABSTRACT

Methods, systems, and apparatus, including computer programs encoded on computer storage media, for a wearable biometric sensor system including a flow rate sensor, a carbon dioxide sensor, a housing configured to retain the flow rate sensor and the carbon dioxide sensor and align the flow rate sensor and carbon dioxide sensor with respect to an air flow path when the housing is affixed to a wearable device, and a control unit in data communication with the flow rate sensor and carbon dioxide sensor.

### TECHNICAL FIELD

This specification relates to wearable sensor devices for measuring a respiratory exchange ratio of a user.

### BACKGROUND

A respiratory exchange ratio (RER) between a volume of carbon dioxide (CO₂) being produced by the body and an amount of oxygen (O₂) being consumed by the body at a point of time (e.g., first thing in the morning) can serve as an indicator of the person's metabolic rate (e.g., a basal metabolic rate), and can be utilized to determine a main fuel source utilized by the body at that time.

### SUMMARY

The technology of this patent application relates to a wearable sensor device for measuring a respiratory exchange ratio of a user. More particularly, the technology includes sensor(s) to measure a flow rate and timing of a user's inhale, exhale, and a concentration of carbon dioxide (CO₂) in the inhale and exhale. The sensor can be incorporated into a wearable device (e.g., a smart watch or another wearable peripheral), or into another mobile device (e.g., a mobile phone, tablet, an attachment for a mobile device such as a case, etc.). The sensor can be positioned on a wearable device such that a user can perform a set of respiratory cycles (i.e., a set of inhale/exhale breaths) over a period of time (or a number of cycles) through the sensor while wearing/holding the wearable device. In some examples, a wearable sensor device is located on a watchband of a smart watch such that the user holds their arm including the watch up to their mouth to perform the measurement.

In some embodiments, the wearable sensor device includes a MEMS-based microphone and a CO₂ sensor configured within a tube such that when a user inhales/exhales through the tube, a flow rate of air is measured by the MEMS-based microphone by measuring a pressure differential between from front and back sides of a diaphragm of the MEMs-based microphone. In some embodiments, the wearable sensor device includes a pair of pressure sensors with a calibrated orifice and a CO₂ sensor configured within a tube such that when a user inhales/exhales through the tube, a flow rate of air is measured by the pair of pressure sensors as difference of pressures between the pair of pressure sensors.

The wearable sensor device can include a control unit (e.g., microcontroller) and/or can provide some or all of signals measured by the wearable sensor device to a processor via a network (e.g., provide voltage signal output to a processor of the smart watch via Bluetooth). In some embodiments, a user's basal metabolic rate can be determined based on the set of respiratory cycles performed by the user through the wearable sensor device. An average exhale flow rate, time of exhale, and concentration of CO₂, an average inhale flow rate, time of inhale, and/or concentration of CO₂ can be determined over the set of respiratory cycles. From the flow rate and time to perform the inhale breath, a volume of oxygen can be determined (assuming an oxygen concentration of 22%). A respiratory exchange ratio is extracted from the average volume of oxygen and average volume of CO₂ over the set of respiratory cycles. A look-up table can be utilized to correlate a respiratory exchange ratio to a metabolic rate (e.g., a basal metabolic rate if a user is performing the set of respiratory cycles during a morning period).

In general, in a first aspect can be embodied in a wearable biometric sensor system including a flow rate sensor, a carbon dioxide sensor, a housing configured to retain the flow rate sensor and the carbon dioxide sensor and align the flow rate sensor and carbon dioxide sensor with respect to an air flow path when the housing is affixed to a wearable device, and a control unit in data communication with the flow rate sensor and carbon dioxide sensor. The control unit is configured to perform operations including, for each respiratory cycle of multiple respiratory cycles performed by a user through the air flow path, detecting, a first respiratory signal from the flow rate sensor along the air flow path, the first respiratory signal including a first start timestamp, a first stop timestamp, and a first flow rate signal, detecting, a first carbon dioxide concentration signal from the carbon dioxide sensor along the air flow path, detecting, a second respiratory signal from the flow rate sensor along the air flow path, the second respiratory including a second start timestamp, a second stop timestamp, and a second flow rate signal, detecting, a second carbon dioxide concentration signal from the carbon dioxide sensor along the air flow path, and generating, from the first respiratory signal, second respiratory signal, first carbon dioxide concentration, and second carbon dioxide concentration, a respiratory exchange ratio, generating, for the plurality of respiratory cycles performed by the user, an average respiratory exchange ratio for the user; and providing the average respiratory exchange ratio for the user.

Other embodiments of this aspect include corresponding methods, computer systems, apparatus, and computer programs recorded on one or more computer storage devices, each configured to perform the actions of the methods.

The foregoing and other embodiments can each optionally include one or more of the following features, alone or in combination. In particular, one embodiment includes all the following features in combination. In some implementations, the system further includes a filter arranged with respect to the air flow path and configured to reduce a threshold amount of humidity in the air flow path.

In some implementations, the flow rate sensor includes a micro-electromechanical system (MEMS) microphone, where the flow rate signal can be a voltage signal corresponding to a difference in an emitted ultrasonic signal and a detected ultrasonic signal.

In some implementations, the flow rate sensor includes two pressure sensors arranged on opposing sides of a calibrated orifice, where the flow rate signal can be measured as a pressure differential across the two pressure sensors through a membrane with the calibrated orifice.

In some implementations, the control unit is a component of the wearable device, and wherein the wearable device is in data communication with the control unit via a wireless data communication link. The wearable device can be a smart watch.

In some implementations, the system further includes correlating the average respiratory exchange ratio to a metabolic rate. Correlating the average respiratory exchange ratio to a metabolic rate can include utilizing a look-up table or a calibrated curve chart or providing the average respiratory exchange ratio to a secondary neural network.

In some implementations, the multiple respiratory cycles include a first set of respiratory cycles captured for the user at rest, and a second set of respiratory cycles captured for the user at a second, different time.

In some implementations, providing the average respiratory exchange ratio for the user includes providing average respiratory exchange ratio and/or metabolic rate to user in a user interface on a user device.

In some implementations, providing the average respiratory exchange ratio for the user includes providing average respiratory exchange ratio and/or metabolic rate for display on the wearable device.

Among other advantages, embodiments can utilize a small-form (low-profile), relatively inexpensive MEMS microphone device (or pair of pressure sensors) that can be integrated within the form factor of a wearable smart device.

Other applications of this technology include, for example, utilizing the wearable sensor device to determine an effect of a particular food on a user's diet (e.g., maintaining ketosis during a ketogenic diet). The technology can be utilized to monitor a user's oxygen inhale volume in real-time (or monitor long-term over many accumulated measurements), for example, to monitor for inflammation, respiratory illness, oxygen consumption efficiency (VO₂ levels).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A is a schematic diagram showing an example operating environment for an example wearable biometric sensor.

FIG. 1B is a schematic diagram showing another example operating environment for another wearable biometric sensor.

FIG. 2 is a flow diagram of an example process for measuring biometric data by a wearable biometric sensor.

FIG. 3 is a block diagram of an example computer system.

Like reference numbers and designations in the various drawings indicate like elements.

### DETAILED DESCRIPTION

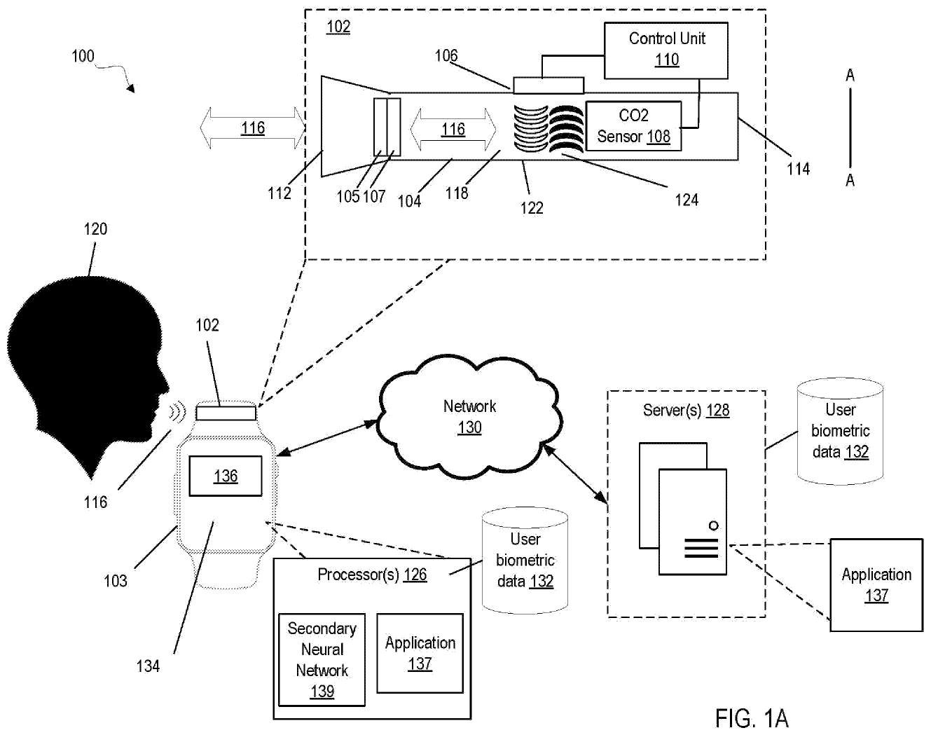

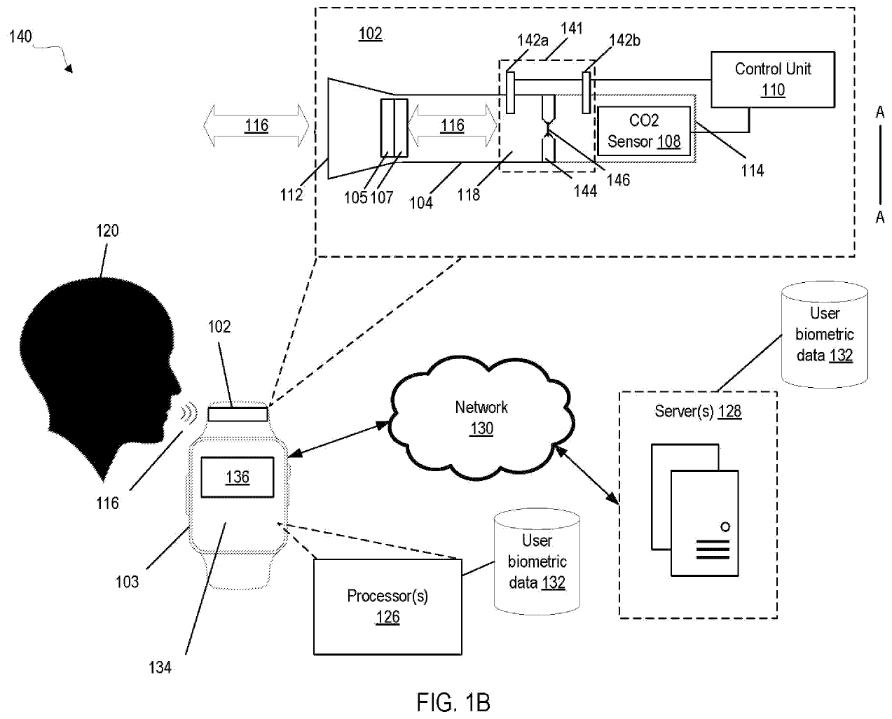

FIG. 1A is an example operating environment 100 for a wearable biometric sensor 102. A wearable biometric sensor 102 includes a housing 104 and a flow rate sensor 106, e.g., a MEMS-based microphone device, and a carbon dioxide (CO₂) sensor 108 both in data communication with a control unit 110. Wearable biometric sensor 102 can be affixed to a smart device 103, e.g., a smart watch, mobile device, tablet, etc. In one example, the wearable biometric sensor 102 is affixed to a watchband of a smart watch.

Housing 104 includes an inlet 112 and an outlet 114 which allow for air flow 116 across the flow rate sensor 106 and CO₂ sensor 108 within a volume 118 of the housing 104. In some implementations, a portion of the housing 104, e.g., a portion of the inlet 112 can be a mouthpiece to accommodate a mouth of a user 120.

A user 120 can inhale/exhale into the inlet 112 to generate air flow 116 through the volume 118 within housing 104. The flow rate sensor 106 is retained within housing 104 such that it is affixed to the housing 104 and arranged within the volume 118 such that air flow 116 is incident on the flow rate sensor 106.

In some implementations, biometric sensor 102 includes a filter 105 arranged and affixed within housing 104 and in-line with air flow 116. Filter 105 can be configured to reduce a threshold amount of humidity in air flow 116 and/or remove particulates or other contaminants in the air flow 116. In some implementations, the biometric sensor 102 can additionally or alternatively include a hygrometer, thermometer, or another environmental measurement sensor to measure humidity, temperature, etc., in the air flow 116 and/or in the environment surrounding the biometric sensor 102. Environmental measurement sensors 107 can be in data communication with control unit 110, and can provide environmental data, e.g., relative humidity, concentration of vapor in air flow 116, temperature, etc., to control unit 110.

In some implementations, as depicted in FIG. 1A, the flow rate sensor 106 is a micro-electromechanical system (MEMS)-based microphone device and configured to emit sound waves 122 (e.g., ultrasonic sound waves) into the volume 118 of the housing 104 and substantially perpendicular (i.e., along axis AA) to an air flow 116 through the volume 118. The flow rate sensor 106, e.g., MEMS-based microphone device, can detect reflected sound waves, i.e., sound waves reflecting off an inner surface of the volume 118 of the housing 104. When air flow 116 is present within the volume 118, e.g., air flow generated by a user inhaling/exhaling through inlet 112, the sound waves 122 emitted by the flow rate sensor 106 interact with the air flow 116 and generate reflected sound waves 124. A flow velocity for the air flow 116 can be measured as a difference in ultrasonic signal between the emitted ultrasonic signal (sound waves 122) and the detected ultrasonic signal (reflected sound waves 124) (i.e., via the Doppler Effect). In some implementations, a direction of air flow 116 (e.g., inhale or exhale) can be determined using at least two sensors placed a known distance apart within housing 104. A time difference between when each sensor detects a same frequency shift between the sound waves 122 and reflected sound waves 124 (which maps to a flow speed) as well as the distance between the sensors can be used to determine a flow direction of air flow 116. In other words, whichever of the two or more sensors that detects a frequency shift first is the sensor of the two or more sensors that is closest to where the air flow 116 has entered the volume 118 of the housing 104.

A control unit 110 in data communication with the flow rate sensor 106 can be configured to operate the flow rate sensor 106 (e.g., provide power, control instructions, etc.) to generate the sound waves 122 and to detect a flow rate signal from the flow rate sensor 106. In some implementations, control unit 110 detects a flow rate signal including reflected sound waves 124, e.g., as an analog voltage signal. The control unit 110 can be configured to collect user biometric data 132, e.g., output signal (analog signal), from the flow rate sensor 106. User biometric data 132 collected from the flow rate sensor 106 can include flow rates corresponding to inhalation and exhalation by the user 120 through the housing 104 of the biometric sensor 102.

Wearable biometric sensor 102 includes a CO₂ sensor 108 retained within the housing 104 such that it is affixed within the volume 118 of the housing 104 and arranged to collect CO₂ concentration measurements of the air flow 116 incident on the CO₂ sensor 108. CO₂ sensor is in data communication with control unit 110 such that the control unit 110 collects a signal output of the CO₂ sensor 108, e.g., an analog voltage signal, indicative of a concentration of CO₂ in the air flow 116 within housing 104.

In some implementations, the control unit 110 is configured to collect user biometric data 132, e.g., signal output from the CO₂ sensor 108 during an exhale of the user through the volume 118 of the housing 104. In other words, the control unit 110 collects data from the CO₂ sensor 108 indicative of the concentration of CO₂ in the user's exhale.

Control unit 110 is further configured to generate/record timestamps, e.g., start/stop times for a detected air flow 116 through the volume 118 of the housing 104. Control unit 110 is configured to register timestamps, e.g., start/stop times for a detected air flow, for example, based on an output signal (in other words, a flow rate signal) from flow rate sensor 106, where a start/stop of the air flow 116 can be registered by how the flow rate sensor measures air flow 116. In some implementations, control unit 110 can detect a threshold change in output signal from the flow rate sensor 106 (e.g., a threshold change in analog voltage signal) as an indicator of an air flow 116 that is an exhale/inhale by user 120.

An example process for assigning timestamps demarcating the initiation/termination of inhale/exhale air flow by a user through the housing 104 includes: control unit 110 detects a threshold change in output signal from the flow rate sensor 106 as an initiation of an inhale air flow 116 by the user 120 and assign a first timestamp T1. Subsequently, control unit 110 can detect a threshold change in output signal from the flow rate sensor 106 as a termination of the inhale air flow 116 by the user 120 and assign a second timestamp T2. The control unit 110 can then detect a threshold change in output signal from the flow rate sensor 106 as an initiation of an exhale air flow 116 by the user 120 and assign a third timestamp T3. Subsequently, control unit 110 can detect a threshold change in output signal from the flow rate sensor 106 as a termination of the exhale air flow 116 by the user 120 and assign a fourth timestamp T4.

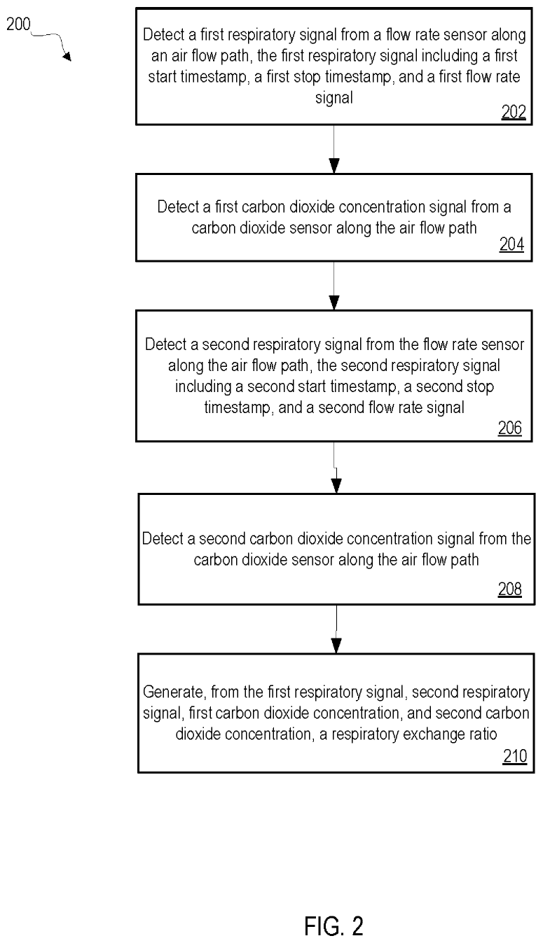

In some implementations, user biometric data 132, e.g., duration and volume of inhale/exhale by the user and CO₂ concentration measurements through the biometric sensor 102, is collected by the control unit 110 for multiple respiratory cycles (e.g., multiple sequential inhale/exhales) to generate an average respiratory exchange ratio. Further discussion of the analysis of collected user biometric data 132 is found below with reference to FIG. 2.

Control unit 110 can be in data communication with one or more processors 126 of a smart device, e.g., smart watch, mobile device, tablet, etc., via a wireless data communication link (e.g., Bluetooth, Wi-Fi, Zigbee, etc.). User biometric data 132 collected by control unit 110, e.g., timestamps, output signals from flow rate sensor 106, CO₂ sensor data, etc., can be provided by the control unit 110 to one or more processors 126 of the smart device 103. In some implementations, some or all of the processes described herein can be performed by the one or more processors 126 of the smart device 103.

In some implementations, control unit 110 can alternatively/additionally be in data communication with one or more cloud-based servers 128 via a network 130. Some or all of the processes described herein can be performed by the one or more cloud-based servers 128 including, for example, logging/analyzing user biometric data over time. User biometric data 132 can be stored locally on the smart device 103 and/or on a cloud-based server 128. User biometric data 132 includes, for example, data collected by the control unit 110 from flow rate sensor 106, CO₂ sensor 108, and/or other sensors of the smart device 103.

In some implementations, control unit 110 includes an analog-to-digital (ADC) converter. The ADC converter can convert analog output signals from the flow rate sensor 106 and/or CO₂ sensor 108 to respective digital output signals. In some implementations, control unit 110 can provide analog voltage signals to an ADC that is a component of the smart device 103, e.g., a component of a smart watch, to convert the analog signal to a digital signal.

Biometric sensor 102 can be affixed to the smart device 103, e.g., on a watchband of a smart watch, and arranged on the smart device 103 such that the inlet 112 of the housing 104 is accessible by the user 120 to provide respiration (i.e., inhale/exhale breathes via the inlet 112 of the housing 104). For example, the biometric sensor 102 can be affixed to a watchband of a smart watch and arranged such that a user 120 of the smart watch can raise the watchband to their mouth and provide respiratory cycles through the biometric sensor while wearing the smart watch.

In some implementations, biometric sensor 102 can be a standalone device and affixed, for example, to a fob, necklace, keychain, etc., for ease of carrying the biometric sensor 102. Control unit 110 of a biometric sensor 102 as a standalone device can include wireless communication functionality, e.g., a Wi-Fi and/or Bluetooth connectivity, to allow the biometric sensor 102 to communicate with a smart device 103 (e.g., a user's smart phone) via wireless communication. Control unit 110 of a biometric sensor 102 as a standalone device can include storage capacity, e.g., solid state memory storage capacity, to store user biometric data 132 locally.

In some implementations, biometric sensor 102 can be affixed to a smart phone or tablet, e.g., via a fixture or adhesive. Biometric sensor 102 can be affixed to a case retaining the smart phone or tablet, e.g., embedded as a part of the case for the smart phone or tablet.

Smart device 103 can include a display 134 via which the user 120 can interact with the biometric sensor 102, e.g., a touch screen, spoken commands, etc. Display 134 can present a user interface 136 of an application 137 to the user 120 including information from the collected user biometric data 132, e.g., average respiratory rate, metabolic rate, and other information related to a user's determined metabolic status (e.g., if the user is in ketosis, what a main source of caloric energy is being burned, etc.). Further details regarding analysis of user biometric data and providing information are discussed below with reference to FIG. 2.

In some implementations, an application 137 operating on the one or more processors 126 and/or on a cloud-based server 128 can receive user biometric data 132 as input and perform one or more calculations using the user biometric data 132 to generate information from the user biometric data 132. For example, application 137 can determine an average respiratory exchange ratio from user biometric data 132 including multiple respiratory cycles by the user 120, in other words, multiple inhale/exhale breaths by the user through the biometric sensor 102.

In some implementations, an average respiratory exchange ratio is calculated utilizing multiple respiratory exchange cycles. For example, application 137 can determine an average respiratory exchange ratio for the user 120 from the user biometric data 132 including multiple respiratory cycles and CO₂ concentration measurements collected from the CO₂ sensor during the multiple respiratory cycles. The application 137 can determine the average respiratory exchange ratio and determine an error value for the average respiratory exchange ratio. A process for determining the average respiratory exchange ratio is described with reference to FIG. 2 below.

In some implementations, application 137 can determine, from the average respiratory exchange ratio, a metabolic rate, e.g., a basal metabolic rate for a user 120 at rest. In one example, application 137 can access a look-up table (LUT) and/or a calibrated curve chart including values for metabolic rate or ranges of metabolic rate corresponding to average respiratory exchange ratios or ranges of average respiratory exchange ratios.

In some implementations, application 137 can utilize a secondary neural network 139 to generate a prediction including a metabolic rate from the average respiratory exchange ratio. The secondary neural network can receive the average respiratory exchange ratio and/or user biometric data 132 as input and generate a prediction including a metabolic rate for the user 120 as output.

FIG. 1B is another example operating environment 140 for a wearable biometric sensor. In some implementations, as depicted in FIG. 1B, a flow rate sensor 141 includes two pressure sensors 142a and 142b arranged with respect to a calibrated orifice 144 including a membrane 146 such that air flow 116 within the volume 118 of the housing 104 of the biometric sensor 102 flows past a first pressure sensor 142a, through the membrane 146 of the calibrated orifice 144, and past a second pressure sensor 142b. The flow rate signal for flow rate sensor 141 is a pressure differential measured across the pressure sensors 142a and 142b through the membrane 146 with the calibrated orifice 144. A flow of air across the flow rate sensor 141 will go from high pressure to low pressure, where the pressure sensor of the two pressure sensors 142a and 142b that measures the higher pressure is the pressure sensor at the side of the orifice 144 where the air flow 116 originated. The calibrated orifice 144 includes a cross-sectional area A₀ arranged perpendicular to an air flow 116 through the inner volume 118 of the housing 104, where air flow 116 through orifice 114 is defined as:

$$Q = {C}_{f} {A}_{0} \sqrt{\frac{2 Δ p}{ρ}} \qquad ( 1 )$$

Where C_{f} is a constant based on the Reynolds Number, Δₚ is a pressure difference between pressure sensors 142a and 142b, and ρ is the density of fluid (air assuming ambient humidity or based on humidity measurements).

Each of the pressure sensors 142a and 142b of the flow rate sensor 141 is in data communication with the control unit 110 such that pressure data, e.g., an analog voltage signal, measured by each pressure sensor 142a and 142b is collected by the control unit 110. As described with reference to FIG. 1A, control unit 110 can collect user biometric data 132, e.g., pressure data from the pressure sensors 142a and 142b, and provide the user biometric data 132, e.g., output analog voltage signal, to the one or more processors 126 of the smart device 103.

Control unit 110 is further configured to generate/record timestamps, e.g., start/stop times for a detected air flow 116 through the volume 118 of the housing 104. Control unit is configured to register timestamps, e.g., start/stop times for a detected air flow, for example, based on an output signal from flow rate sensor 141, where a start/stop of the air flow 116 can be registered by how the flow rate sensor measures air flow 116. For example, control unit 110 can detect a threshold change in pressure data (e.g., analog voltage signal) generated by one or both of the pressure sensors 142a and 142b. In some implementations, control unit 110 can detect a threshold change in output signal from the flow rate sensor 141 (e.g., a threshold change in analog output) as an indicator of an air flow 116 that is an exhale/inhale by user 120.

An example process for assigning timestamps demarcating the initiation/termination of inhale/exhale air flow by a user through the housing 104 includes: control unit 110 detects a threshold change in output signal from the flow rate sensor 141 as an initiation of an inhale air flow 116 by the user 120 and assign a first timestamp T1. Subsequently, control unit 110 can detect a threshold change in output signal from the flow rate sensor 141 as a termination of the inhale air flow 116 by the user 120 and assign a second timestamp T2. The control unit 110 can then detect a threshold change in output signal from the flow rate sensor 141 as an initiation of an exhale air flow 116 by the user 120 and assign a third timestamp T3. Subsequently, control unit 110 can detect a threshold change in output signal from the flow rate sensor 141 as a termination of the exhale air flow 116 by the user 120 and assign a fourth timestamp T4.

FIG. 2 is a flow diagram of an example process 200 for measuring biometric data by a wearable biometric sensor. As described above, the processes described with reference to FIG. 2 can be performed by the one or more processors 126, a cloud-based server 128, or a combination thereof. A biometric sensor 102 (i.e., a control unit 110 of the biometric sensor 102) detects a first respiratory signal from a flow rate sensor, e.g., flow rate sensor 106/141, along an air flow 116 within a volume 118 of housing 104 of the biometric sensor 102, where the first respiratory signal includes a first start timestamp, a first stop timestamp, and a first flow rate signal (202). The first respiratory signal can be representative of a first respiratory event, e.g., inhalation or exhalation by the user 120 through the biometric sensor 102.

In some implementations, control unit 110 detects a first respiratory signal, e.g., a threshold change in output signal from flow rate sensor 106/141 representative of air flow 116 through the housing 104. A first start timestamp is generated to designate when the control unit 110 detects an initiation of the first respiratory signal at the flow rate sensor 106, 141 (e.g., a first threshold change in output signal from the flow rate sensor) and a first stop timestamp is generated to designate when the control unit 110 detects a termination of the first respiratory signal at the flow rate sensor 106, 141 (e.g., a second threshold change in output signal from the flow rate sensor).

The biometric sensor 102 detects a first carbon dioxide (CO₂) concentration signal from a carbon dioxide sensor 108 along the air flow path (204). In some implementations, control unit 110 detects a first CO₂ concentration signal, i.e., an analog voltage signal, from the CO₂ sensor 108 representative of a detected concentration of CO₂ in the air flow 116 during the first respiratory event (e.g., inhalation or exhalation).

The biometric sensor 102 detects a second respiratory signal from the flow rate sensor along the air flow path, where the second respiratory signal includes a second start timestamp, a second stop timestamp, and a second flow rate signal (206). The second respiratory signal can be representative of a second respiratory event, e.g., inhalation or exhalation by the user 120 through the biometric sensor 102.

In some implementations, control unit 110 detects a second respiratory signal, e.g., a threshold change in output signal from flow rate sensor 106/141 representative of air flow 116 through the housing 104. A second start timestamp is generated to designate when the control unit 110 detects an initiation of the second respiratory signal at the flow rate sensor 106/141 (e.g., a first threshold change in output signal from the flow rate sensor) and a second stop timestamp is generated to designate when the control unit 110 detects a termination of the second respiratory signal at the flow rate sensor 106/141 (e.g., a second threshold change in output signal from the flow rate sensor).

The biometric sensor 102 detects a second carbon dioxide concentration signal from the carbon dioxide sensor 108 along the air flow path (208). In some implementations, control unit 110 detects a second CO₂ concentration signal, i.e., an analog voltage signal, from the CO₂ sensor 108 representative of a detected concentration of CO₂ in the air flow 116 during the second respiratory event (e.g., inhalation or exhalation). The relative CO₂ concentrations of the first respiratory event and the second respiratory event can be used to determine inhale vs exhale, e.g., where the exhale will have a higher concentration of CO₂ and the inhale will have a lower concentration of CO₂.

A respiratory exchange ratio is calculated, e.g., by one or more processors 126 of a smart device 103, by a cloud-based server 128, or a combination thereof, from the first respiratory signal, second respiratory signal, first carbon dioxide concentration, and second carbon dioxide concentration (210).

In some implementations, the first respiratory signal, second respiratory signal, first CO₂ concentration signal and second CO₂ concentration signal are collected by the control unit 110 as voltages from the respective devices (i.e., the flow rate sensor 106, 141 and CO₂ sensor 108) which are subsequently converted to digital signals (e.g., by an ADC located at the biometric sensor 102 or downstream at the smart device 103).

A volume of CO₂ in an exhalation of the user 120 can be calculated by:

V(E)_{CO2}=F_{E}*(T₂−T₁)*C(E)_{CO2} (2)

where V(E)_{CO2} is the volume of CO₂ in the exhalation, F_{E} is the flow rate of exhalation, T₂, T₁ are timestamps for the stop of exhalation and start of exhalation, respectively, and C(E)_{CO2} is the concentration of CO₂ in the exhalation as measured by the CO₂ sensor.

A volume of CO₂ in an inhalation of the user 120 can be calculated by:

V(I)_{CO2}=F_{I}*(T₄−T₃)*C(I)_{CO2} (3)

where V(I)_{CO2} is the volume of CO₂ in the inhalation, F_{I} is the flow rate of inhalation, T₄, T₃ are timestamps for the stop of inhalation and start of inhalation, respectively, and C(I)_{CO2} is the concentration of CO₂ in the inhalation as measured by the CO₂ sensor.

A volume of oxygen (O₂) in an inhalation of the user 120 can be calculated by:

V(I)_{O2}=F_{I}*(T₄−T₃)*C(I)_{O2} (4)

where V(I)_{O2} is the volume of O₂ in the inhalation, F_{I} is the flow rate of inhalation, T₄, T₃ are timestamps for the stop of inhalation and start of inhalation, respectively, and C(I)_{O2} is the concentration of O₂ in the inhalation. A concentration of O₂ in inhalation can be assumed to be 22%, based on average O₂ levels in air.

A respiratory exchange ratio (RER) can be calculated by:

RER=V(E)_{CO2}/V(I)_{O2} (5)

An average RER can be calculated by averaging multiple RER values for respective respiratory cycles collected by the biometric sensor 102. For example, a user 120 can perform a set of five respiratory cycles through the biometric sensor which can be utilized to calculate an average RER for the user.

In some implementations, a metabolic rate value is calculated from the average RER. As described above, application 137 can utilize a LUT or a secondary neural network to correlate the average RER with a metabolic rate value (or range of metabolic rate values), e.g., between 0.7-1. The LUT can be utilized to output a metabolic rate (unit energy per unit time), e.g., in watts, Joules per hour per kg body mass, etc. The metabolic rate value can be further correlated (e.g., using a LUT) with burn rates of fat/carbs/protein.

In some implementations, the biometric sensor 102 can operate in multiple modes, e.g., to calculate metabolic rate values when the user is at rest or during user activity. The biometric sensor 102 can be utilized to collect multiple sets of respiratory cycles to determine a metabolic rate for the user 120 performing different activities or at different points in the day, for example, a first set of respiratory cycles captured when the user 120 is at rest (e.g., basal metabolic rate) and a second set of respiratory cycles captured for the user at a second, different time (e.g., while the user 120 is exercising). The determined metabolic rate value can be used to give the user 120 information related to how the user's body is processing calories (e.g., is in ketosis based on metabolic rate).

In some implementations, the respiratory exchange ratio (RER) value, metabolic rate value, and/or other information related to processing of calories is provided to a user 120. Application 137 can provide information generated from user biometric data 132 collected by the biometric sensor 102 for presentation via a user interface 136, e.g., on the display 134 of the smart device 103. For example, “Your metabolic rate value indicates that you are within a range for ketosis.”

In some implementations, long-term monitoring can be utilized to alert a user to patterns in their metabolic rate values, e.g., basal metabolic rate over a period of time or to monitor that a user stays in ketosis for an extended period of time.

In some implementations, long-term monitoring can be utilized to determine foods that affect a user's diet. e.g., for a ketogenic diet the metabolic rate should stay below 0.8. Respiratory cycle measurements can be taken before/after eating to determine how foods affect metabolic rate.

In some implementations, a biometric sensor 102 can be utilized to monitor an O₂ inhale volume for monitoring an asthmatic user during an inflammatory event. In some implementations, a biometric sensor 102 can be utilized to monitor RER and/or O₂ inhale volumes for a user experiencing respiratory illness. In some implementations, a biometric sensor 102 can be utilized to monitor O₂ inhale volume levels (VO₂) for runners (e.g., to measure oxygen efficiency).

In some implementations, a biometric sensor can be used in combination with a temperature measurement from another device (e.g., a smart watch including a thermometer to improve the accuracy of the RER and/or metabolic rate value measurements.

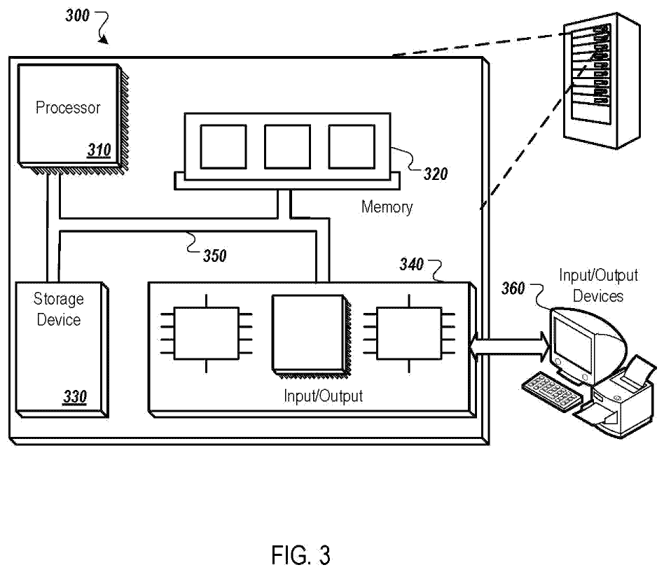

FIG. 3 is block diagram of an example computer system 300 that can be used to perform operations described above. The system 300 includes a processor 310, a memory 320, a storage device 330, and an input/output device 340. Each of the components 310, 320, 330, and 340 can be interconnected, for example, using a system bus 350. The processor 310 is capable of processing instructions for execution within the system 300. In one implementation, the processor 310 is a single-threaded processor. In another implementation, the processor 310 is a multi-threaded processor. The processor 310 is capable of processing instructions stored in the memory 320 or on the storage device 330.

The memory 320 stores information within the system 300. In one implementation, the memory 320 is a computer-readable medium. In one implementation, the memory 320 is a volatile memory unit. In another implementation, the memory 320 is a non-volatile memory unit.

The storage device 330 is capable of providing mass storage for the system 300. In one implementation, the storage device 330 is a computer-readable medium. In various different implementations, the storage device 330 can include, for example, a hard disk device, an optical disk device, a storage device that is shared over a network by multiple computing devices (for example, a cloud storage device), or some other large capacity storage device.

The input/output device 340 provides input/output operations for the system 300. In one implementation, the input/output device 340 can include one or more network interface devices, for example, an Ethernet card, a serial communication device, for example, and RS-232 port, and/or a wireless interface device, for example, and 302.11 card. In another implementation, the input/output device can include driver devices configured to receive input data and send output data to other input/output devices, for example, keyboard, printer and display devices 360. Other implementations, however, can also be used, such as mobile computing devices, mobile communication devices, set-top box television client devices, etc.

Although an example processing system has been described in FIG. 3, implementations of the subject matter and the functional operations described in this specification can be implemented in other types of digital electronic circuitry, or in computer software, firmware, or hardware, including the structures disclosed in this specification and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, that is, one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, for example, a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, for example, an FPGA (field programmable gate array) or an ASIC (application-specific integrated circuit). The apparatus can optionally include, in addition to hardware, code that creates an execution environment for computer programs, for example, code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, for example, an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a Global Positioning System (GPS) receiver, or a portable storage device, for example, a universal serial bus (USB) flash drive, to name just a few.

Computer-readable media suitable for storing computer program instructions and data include all forms of non-volatile memory, media and memory devices, including by way of example semiconductor memory devices, for example, EPROM, EEPROM, and flash memory devices; magnetic disks, for example, internal hard disks or removable disks; magneto-optical disks; and CD-ROM and DVD-ROM disks.

To provide for interaction with a user, embodiments of the subject matter described in this specification can be implemented on a computer having a display device, for example, a CRT (cathode ray tube) or LCD (liquid crystal display) monitor, for displaying information to the user and a keyboard and a pointing device, for example, a mouse or a trackball, by which the user can provide input to the computer. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, for example, a smartphone that is running a messaging application, and receiving responsive messages from the user in return.

Data processing apparatus for implementing machine learning models can also include, for example, special-purpose hardware accelerator units for processing common and compute-intensive parts of machine learning training or production, that is, inference, workloads.

Machine learning models can be implemented and deployed using a machine learning framework, for example, a TensorFlow framework, a Microsoft Cognitive Toolkit framework, an Apache Singa framework, or an Apache MXNet framework.

Embodiments of the subject matter described in this specification can be implemented in a computing system that includes a back-end component, for example, as a data server, or that includes a middleware component, for example, an application server, or that includes a front-end component, for example, a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, for example, a communication network. Examples of communication networks include a local area network (LAN) and a wide area network (WAN), for example, the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, for example, an HTML page, to a user device, for example, for purposes of displaying data to and receiving user input from a user interacting with the device, which acts as a client. Data generated at the user device, for example, a result of the user interaction, can be received at the server from the device.

While this specification contains many specific implementation details, these should not be construed as limitations on the scope of any features or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A wearable biometric sensor system comprising:
a flow rate sensor;
a carbon dioxide sensor;
a housing configured to retain the flow rate sensor and the carbon dioxide sensor and align the flow rate sensor and carbon dioxide sensor with respect to an air flow path when the housing is affixed to a wearable device; and
a control unit in data communication with the flow rate sensor and carbon dioxide sensor, wherein the control unit is configured to perform operations comprising:
for each respiratory cycle of a plurality of respiratory cycles performed by a user through the air flow path:
detecting, a first respiratory signal from the flow rate sensor along the air flow path, the first respiratory signal comprising a first start timestamp, a first stop timestamp, and a first flow rate signal;
detecting, a first carbon dioxide concentration signal from the carbon dioxide sensor along the air flow path;
detecting, a second respiratory signal from the flow rate sensor along the air flow path, the second respiratory comprising a second start timestamp, a second stop timestamp, and a second flow rate signal;
detecting, a second carbon dioxide concentration signal from the carbon dioxide sensor along the air flow path; and
generating, from the first respiratory signal, second respiratory signal, first carbon dioxide concentration, and second carbon dioxide concentration, a respiratory exchange ratio;
generating, for the plurality of respiratory cycles performed by the user, an average respiratory exchange ratio for the user; and
providing the average respiratory exchange ratio for the user.

2. The system of claim 1, further comprising a filter arranged with respect to the air flow path and configured to reduce a threshold amount of humidity in the air flow path.

3. The system of claim 1, wherein the flow rate sensor comprises a micro-electromechanical system (MEMS) microphone.

4. The system of claim 3, wherein the flow rate signal is a voltage signal corresponding to a difference in an emitted ultrasonic signal and a detected ultrasonic signal.

5. The system of claim 1, wherein the flow rate sensor comprises two pressure sensors arranged on opposing sides of a calibrated orifice.

6. The system of claim 5, wherein the flow rate signal comprises a pressure differential measured across the two pressure sensors through a membrane with the calibrated orifice.

7. The system of claim 1, wherein the control unit is a component of the wearable device, and wherein the wearable device is in data communication with the control unit via a wireless data communication link.

8. The system of claim 1, wherein the wearable device is a smart watch.

9. The system of claim 1, further comprising correlating the average respiratory exchange ratio to a metabolic rate.

10. The system of claim 9, wherein correlating the average respiratory exchange ratio to a metabolic rate comprises utilizing a look-up table or a calibrated curve chart.

11. The system of claim 9, wherein correlating the average respiratory exchange ratio to a metabolic rate comprises providing the average respiratory exchange ratio to a secondary neural network.

12. The system of claim 1, wherein the plurality of respiratory cycles comprise:
a first set of respiratory cycles captured for the user at rest; and
a second set of respiratory cycles captured for the user at a second, different time.

13. The system of claim 12, wherein providing the average respiratory exchange ratio for the user comprises providing average respiratory exchange ratio and/or metabolic rate to user in a user interface on a user device.

14. The system of claim 13, wherein providing the average respiratory exchange ratio for the user comprises providing average respiratory exchange ratio and/or metabolic rate for display on the wearable device.

15. A method for a wearable biometric sensor comprising:
receiving, from a flow rate sensor and a carbon dioxide sensor arranged with respect to an air flow path of the wearable biometric sensor affixed to a wearable device, a plurality of respiratory cycles performed by a user through the air flow path;
for each respiratory cycle of the plurality of respiratory cycles performed by the user through the air flow path:
detecting, a first respiratory signal from the flow rate sensor along the air flow path, the first respiratory signal comprising a first start timestamp, a first stop timestamp, and a first flow rate signal;
detecting, a first carbon dioxide concentration signal from the carbon dioxide sensor along the air flow path;
detecting, a second respiratory signal from the flow rate sensor along the air flow path, the second respiratory comprising a second start timestamp, a second stop timestamp, and a second flow rate signal;
detecting, a second carbon dioxide concentration signal from the carbon dioxide sensor along the air flow path; and
generating, from the first respiratory signal, second respiratory signal, first carbon dioxide concentration, and second carbon dioxide concentration, a respiratory exchange ratio;
generating, for the plurality of respiratory cycles performed by the user, an average respiratory exchange ratio for the user; and
providing the average respiratory exchange ratio for the user.

16. The method of claim 15, further comprising:
determining an inhaled oxygen volume based on the first respiratory signal and the second respiratory signal.

17. The method of claim 15, further comprising:
determining a first exhaled volume of CO₂ by the user and a second exhaled volume of CO₂ by the user from the first carbon dioxide concentration signal, second carbon dioxide signal.

18. The method of claim 15, further comprising correlating the average respiratory exchange ratio to a metabolic rate.

19. The method of claim 18, wherein correlating the average respiratory exchange ratio to a metabolic rate comprises utilizing a look-up table or a calibrated curve chart.

20. The method of claim 18, wherein correlating the average respiratory exchange ratio to a metabolic rate comprises providing the average respiratory exchange ratio to a secondary neural network.
